(19) United States
(12) Patent Application Publication
(10) Pub. No.: US 2023/0037256 A1
(43) Pub. Date: Feb. 2, 2023

(54) ENDOSCOPE HAVING ILLUMINATED HANDLE

(71) Applicant: Gyrus ACMI, INC. D/B/A Olympus Surgical Technologies America, Westborough, MA (US)

(72) Inventors: Peter J. Crowley, Norfolk, MA (US); Jordan C. Avalos, Littleton, MA (US)

(21) Appl. No.: 17/814,645

(22) Filed: Jul. 25, 2022

Related U.S. Application Data
(60) Provisional application No. 63/203,534, filed on Jul. 27, 2021.

(51) Int. Cl. A61B 1/00; A61B 1/05; A61B 1/06; A61B 1/07; A61B 1/018

(52) U.S. Cl. CPC A61B 1/00066; A61B 1/05; A61B 1/0669; A61B 1/07; A61B 1/018; A61B 2090/0807

(57) ABSTRACT

An endoscope body of an endoscope can include a handle, a shaft extending distally from the handle, and a distal portion at a distal end of the shaft. The handle can include a light guide that can receive illumination from a light source that is separate from the endoscope body. The light guide can include an illumination splitter that can split the received illumination into a target illumination and a handle illumination. The light guide can propagate the target illumination to the distal portion to illuminate a target. The distal portion can optionally include a camera that can capture an image of the illuminated target. The illumination splitter can propagate the handle illumination to illuminate at least a portion of the handle, such as an instrument port that can provide access to a proximal end of an instrument sheath that extends to the distal portion.

### PRIORITY CLAIMS

This application claims the benefit of priority to U.S. Provisional Patent Application Ser. No. 63/203,534, filed Jul. 27, 2021, the contents of which are hereby incorporated by reference in their entirety.

### FIELD OF THE DISCLOSURE

The present disclosure relates generally to an endoscope.

### BACKGROUND OF THE DISCLOSURE

An endoscope can be introduced into the body of a patient to view specific locations within the patient's body. There is ongoing effort to improve the performance of endoscopes.

### SUMMARY

In an example of an endoscope, an endoscope body can include a handle, a shaft extending distally from the handle, and a distal portion at a distal end of the shaft. The handle can include a light guide that can receive illumination from a light source that is separate from the endoscope body. The light guide can include an illumination splitter that can split the received illumination into a target illumination and a handle illumination. The light guide can propagate the target illumination to the distal portion to illuminate a target. The distal portion can include a camera that can capture an image of the illuminated target. The illumination splitter can further propagate the handle illumination to illuminate at least a portion of the handle.

In an example of a method for operating an endoscope, the endoscope can include a handle, a shaft extending distally from the handle, and a distal portion at a distal end of the shaft. The method can include: receiving illumination from a light source that is separate from the endoscope body; splitting the received illumination into a target illumination and a handle illumination; propagating the target illumination to the distal portion to illuminate a target; and propagating the handle illumination to illuminate at least a portion of the handle.

In an example of an endoscope, an endoscope body can include a handle that includes a window, a shaft extending distally from the handle, and a distal portion at a distal end of the shaft. The handle can include a light guide that can receive illumination from a light source that is separate from the endoscope body. The light guide can include a textured portion that can split the received illumination into a target illumination and a handle illumination. The light guide can propagate the target illumination to the distal portion to illuminate a target. The distal portion can include a camera that can capture an image of the illuminated target. The textured portion can propagate the handle illumination toward the window to illuminate at least a portion of the handle.

### BRIEF DESCRIPTION OF THE DRAWINGS

FIG. 1 shows a side view of an example of an endoscope that can receive illumination from an external light source and direct some of the received illumination to illuminate some or all of the handle.

FIG. 2 shows a schematic drawing of an example of the illuminating elements of the endoscope of FIG. 1.

FIG. 3 shows a side view of an example of an illumination splitter.

FIG. 4 shows a side view of an example of an illumination splitter.

FIG. 5 shows a side view of an example of an illumination splitter.

FIG. 6 shows a side view of an example of an illumination splitter.

FIG. 7 shows an example of a method for operating an endoscope.

Corresponding reference characters indicate corresponding parts throughout the several views. Elements in the drawings are not necessarily drawn to scale. The configurations shown in the drawings are merely examples and should not be construed as limiting in any manner.

### DETAILED DESCRIPTION

An endoscope can receive illumination from an external light source (e.g., a light source not included in a handle of the endoscope) and direct some of the received illumination to illuminate some or all of the handle. Compared to an endoscope that includes a light-emitting diode or other light source in the handle, which can generate heat in the handle and may therefore cause discomfort for a practitioner, the endoscope described in detail below can lack such a light source, and can therefore generate less heat or no heat in the handle, and, as a result, can decrease or eliminate discomfort of a practitioner during use of the endoscope.

In an example of an endoscope, an endoscope body can include a handle, a shaft extending distally from the handle, and a distal portion at a distal end of the shaft. The handle can include a light guide that can receive illumination from a light source that is separate from the endoscope body. The light guide can include an illumination splitter that can split the received illumination into a target illumination and a handle illumination. The light guide can propagate the target illumination to the distal portion to illuminate a target. The distal portion can include a camera that can capture an image of the illuminated target. The illumination splitter can further propagate the handle illumination to illuminate at least a portion of the handle.

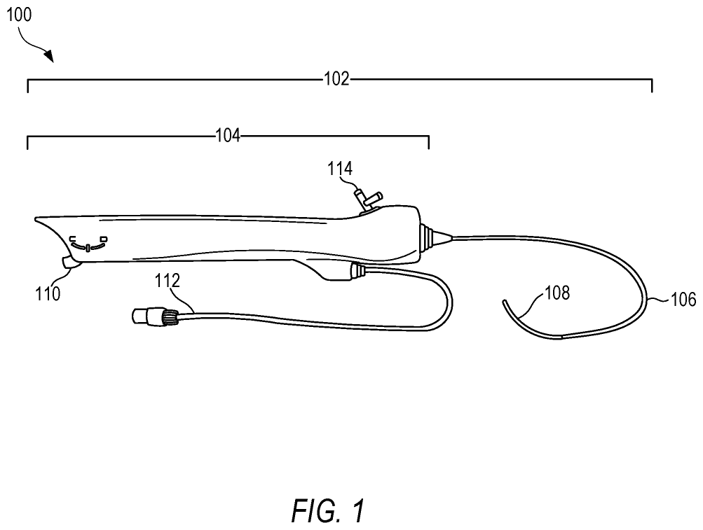

FIG. 1 shows a side view of an example of an endoscope 100 that can receive illumination from an external light source and direct at least some of the received illumination to illuminate some or all of the handle. In some examples, the light source can be disposed and/or located within the handle, attached to the handle, coupled to the handle, disposed on the handle, or otherwise located proximate to the handle or internal to the handle.

The endoscope 100 can include an endoscope body 102. The endoscope body 102 can connect to a controller (not shown), such as a computer. In some examples, the endoscope body 102 can include all the elements of the endoscope 100 that are not part of the controller. For applications in which the endoscope body 102 can operate as a single-use device, the endoscope body 102 can include all the elements of the endoscope 100 that can be discarded at the end of a procedure. For example, the controller may not be part of the endoscope body 102.

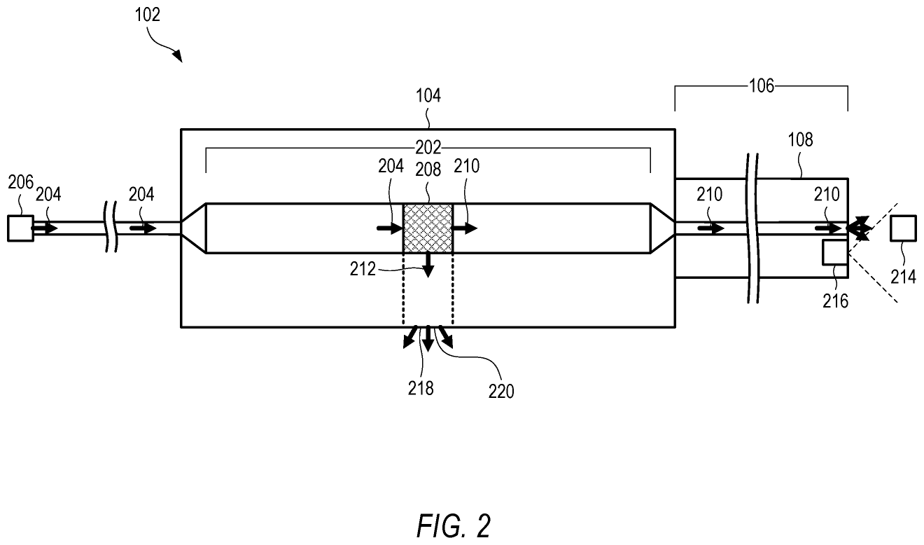

The endoscope body 102 can include a handle 104, a shaft 106 extending distally from the handle 104, and a distal portion 108 at a distal end of the shaft 106. During a procedure, a practitioner can insert the distal portion 108 into the body of a patient. The practitioner can advance the distal portion 108 distally through the body, such as through passages in the lung, or through a series of blood vessels, to reach a target 214 (FIG. 2), such as blood clot in the lungs, or a blockage in a blood vessel. During the procedure, the practitioner can advance one or more instruments, via an instrument port 114, through an instrument sheath that extends through the shaft 106 to the distal portion 108. The practitioner can use the instruments to affect the target 214, such as by ablating the target 214 or cutting the target 214 into smaller pieces. After the instruments have been used, the practitioner can retract the instruments through the instrument sheath. At the end of the procedure, the practitioner can withdraw the distal portion 108 from the body of the patient.

The endoscope 100 can include an actuator (not shown) that can adjust a position of the distal portion 108. The actuator can allow the practitioner to steer the distal portion 108 as needed to direct the distal portion 108 through the body to the target 214. For example, the actuator can include a pair of cables disposed on opposing sides of the shaft 106 and extending along the shaft 106 from the handle 104 to the distal portion 108. The actuator can apply different forces to the cables in the pair, such as advancing one cable distally and retracting the other cable proximally, to move the distal portion 108. This is but one example of an actuator. Other suitable actuators can also be used.

The handle 104 can include a distal portion controller 110, which can cause the actuator to adjust the position of the distal portion 108. For example, the distal portion controller 110 can include a positionable element, such as a lever, a switch, a knob, a button, or others. The practitioner can adjust the distal portion controller 110 to cause the actuator to move the distal portion 108 in a desired manner. For example, the practitioner can move the distal portion controller 110 to actuate the distal portion 108, such as to curl the distal portion 108 in one direction or in an opposing direction. In some examples, the distal portion controller 110 can vary the position of the distal portion 108 in one dimension, such as by causing the distal portion 108 to curl in a selectable position that lies in a single plane. In some examples, the distal portion controller 110 can vary the position the distal portion 108 in two dimensions, such as by using two positionable elements to control movement in orthogonal planes, or using a curl controller to control a curl in a specified plane and an azimuthal controller to rotate the specified plane about a longitudinal axis of the distal portion 108. These are but examples of distal portion controllers 110. Other suitable distal portion controllers 110 can also be used to actuate the distal portion 108 of the endoscope body 102.

The distal portion 108 can include a camera 216 (FIG. 2) that can capture an image of the target 214 (FIG. 2). The camera 216 can include a lens and a sensor disposed at a focal plane of the lens. The sensor can generate a video signal, such as an analog video signal or a digital video signal, that can provide a real-time (or nearly real-time) visualization of a volume that extends conically away from the distal end of the distal portion 108.

The endoscope body 102 can include a connector 112 that can electrically connect the endoscope body 102 to the controller. The connector 112 can include electrical connections that can supply electrical power to the endoscope body 102, such as to power the sensor of the camera 216, and can receive electrical signals, such as the video signal generated by the sensor of the camera 216. The controller can display the video signal on a monitor. The monitor can be integrated into the controller or can be separate from the controller. During use, the monitor can display a video image corresponding to the video signal, which can provide a real-time (or near real-time) visualization of the volume that extends conically away from the distal end of the distal portion 108. During a procedure, the practitioner can view the video image on the monitor, and in response to the viewed video image, can position the handle 104, the shaft 106, and the distal portion controller 110 to guide the distal end toward the target 214.

FIGS. 2-6 and the paragraphs that follow explain how the endoscope body 102 can receive illumination from an external light source and can direct some of the received illumination to illuminate some or all of the handle 104.

FIG. 2 shows a schematic drawing of an example of the illuminating elements of the endoscope 100 of FIG. 1. The endoscope body 102 can receive illumination from an external light source and can direct some of the received illumination to illuminate some or all of the handle 104.

The handle 104 can include a light guide 202, which can guide light via reflection from a light-guiding surface of the light guide 202. The light-guiding surface can include an interface between air and an optical medium, such as glass or plastic. In some examples, the light-guiding surface can guide light inside the light guide 202 via total internal reflection from the light-guiding surface. In some examples, the light guide 202 can optionally include one or more reflective coatings or reflective films disposed on the light-guiding surface. In some examples, the light guide 202 can include an optical fiber, which can be generally flexible and can be routed to take any suitable path within the handle 104. As another example, the light guide 202 can include a solid rod, which can optionally be generally rigid and can be manufactured to have any suitable shape, such that the solid rod can take any suitable path within the handle 104. The fiber and rod are but mere examples. Other suitable light guides 202 can also be used.

The light guide 202 can receive illumination 204 from a light source 206 that is separate from the endoscope body 102. The light source 206 can optionally be included with the controller, or optionally attached to the controller. The light source 206 can include a light-emitting diode, multiple light-emitting diodes, one or more laser diodes, a gas laser, a gas discharge lamp, an incandescent lamp, or any other suitable light-producing element. The light source 206 can emit white light, such as via emission from a white-light light-emitting diode. The illumination 204 can propagate from the light source 206 to the handle 104 via an optical fiber, such as a multi-mode optical fiber or a single-mode optical fiber. In some examples, the optical fiber can be included with the connector 112 (FIG. 1), such that the connector 112 can provide an optical connection for the illumination 204, in addition to the electrical connections for the sensor in the camera 216. In some examples, the optical fiber can be separate from the connector 112, and can include a dedicated connection, separate from the electrical connections, to the handle 104. The endoscope body 102 can optionally include one or more additional elements, such as a beam-expanding lens (not shown), that can increase an efficiency for coupling the illumination 204 into the light guide 202.

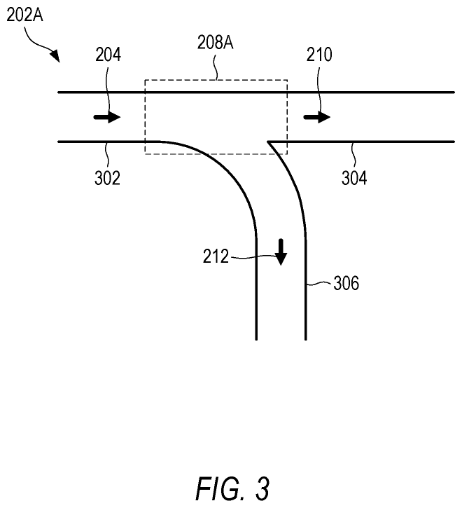

The light guide 202 can include an illumination splitter 208 that can split the received illumination 204 into a target illumination 210 and a handle illumination 212. The light guide 202 can propagate the target illumination 210 to the distal portion 108 to illuminate a target 214. The illumination splitter 208 can further propagate the handle illumination 212 to illuminate at least a portion of the handle 104. By directing a portion of the received illumination 204 to illuminate at least a portion of the handle 104, the endoscope body 102 may avoid disposing a light source in or on the handle 104, and may therefore avoid issues associated with such a light source, such as generating heat that could undesirably heat the handle 104 and cause discomfort for the practitioner during use. In some examples, such as for a single-use and/or disposable device, the absence of a light source in the handle can simplify disposal of the device, such as by including fewer restricted or complex material that would otherwise be included with such a light source. For example, a light-emitting diode can include metal shielding, solder, lead, or other elements that can be difficult to dispose of. FIGS. 3-6 below provide specific examples of suitable illumination splitters 208.

In some examples, the handle 104 can include a window 218 that can pass the handle illumination 212 out of the handle 104. The window 218 can include one or more areas on the handle 104 or can optionally include the entire handle 104. In some examples, the window 218 can be transparent, such an element having smooth surfaces. The handle illumination 212 can propagate through such a transparent window 218 by refracting through the window surfaces without significant scattering or diffusing (e.g., where a single incident ray produces a single exiting ray along a single direction). In other examples, the window 218 can optionally be frosted or textured. The handle illumination 212 can propagate through such a frosted or textured window 218 with scattering or diffusing, which can widen an angular distribution of light that passes through the window 218 (e.g., where a single incident ray produces multiple exiting rays having an angular distribution that can optionally be centered on the ray that would be present if no scattering or diffusion occurred). In some examples, the window 218 can be formed from a thinned opaque material. In some examples, the window 218 can be formed from a different material than other areas of the handle 104.

In some examples, the endoscope body 102 can include an instrument sheath that extends through the shaft 106 (FIG. 1) to the distal portion 108 (FIG. 1). The handle 104 can include an instrument port 114 (FIG. 1) that can provide access to a proximal end of the instrument sheath. The illumination splitter 208 can further propagate the handle illumination 212 to illuminate at least a portion of the instrument port 114 and/or at least partially illuminate a perimeter of the instrument port 114.

In some examples, the distal portion 108 (FIG. 1) is actuatable. The handle 104 (FIG. 1) can include a distal portion controller 110 (FIG. 1) that can actuate the distal portion 108 (FIG. 1). The illumination splitter 208 can further propagate the handle illumination 212 to illuminate at least a portion of the distal portion controller 110 and/or at least partially illuminate a perimeter of the distal portion controller 110.

In some examples, the handle 104 (FIG. 1) can include indicia 220 (FIG. 2). In some examples, the indicia 220 can provide visual instructions for operating the endoscope 100. For example, the indicia 220 can include a symbol that indicates that the endoscope is intended for single use. In some examples, such a symbol can include a numeral 2 with a circle around it and a line through it. Other examples of indicia 220 include an arrow that indicates a particular direction on the handle, an environmentally friendly symbol, a graduated measurement scale such as a ruler having markings at specific locations and/or spaced apart by one or more specified distances, and others. In some examples, the indicia 220 can identify at least one characteristic of the endoscope 100. For example, the indicia 220 can include a name of a product, a brand, a logo, an inventory part number, a lot number, a serial number, a standard configuration for actuating the distal portion, a reverse configuration for actuating the distal portion, and others. The illumination splitter 208 can further propagate the handle illumination 212 to form a backlight for the indicia 220 and/or at least partially illuminate a perimeter of the indicia 220.

FIGS. 3-6 show examples of suitable illumination splitters 208. The configurations of FIGS. 3-6 are but mere examples. Other suitable illumination splitters can also be used.

FIG. 3 shows a side view of an example 208A of an illumination splitter, such as 208 (FIG. 2).

In the configuration of FIG. 3, the light guide 202A is shaped to direct the illumination via total internal reflection from at least one exterior surface of the light guide 202A. For example, the light guide 202A can be formed as an elongated rod or pipe, which can optionally include bends, a spiral shape, or other features that can include bend radii that are large enough to avoid violating the condition of total internal reflection from the exterior surface or exterior surfaces of the light guide 202A.

In the configuration of FIG. 3, the illumination splitter 208A can include a junction in the light guide 202A. The junction can be shaped as a fork, a branch, or other suitable geometry and/or can include any component that can direct light from a single input path into multiple output paths. In some examples, the junction can include a reflective surface, such as a mirrored surface. In some examples, the junction can include a clamped element to divert at least a portion of the light toward the window 218. The light guide 202A can include a received illumination light guide portion 302 that can guide the received illumination 204 to the junction. The light guide 202A can include a target illumination light guide portion 304, extending from the junction, that can guide the target illumination 210 along the target illumination light guide portion 304 via total internal reflection from at least one exterior surface of the target illumination light guide portion 304. The light guide 202A can include a handle illumination light guide portion 306, extending from the junction, that can guide the handle illumination 212 toward the window 218 along the handle illumination light guide portion 306 via total internal reflection from at least one exterior surface of the handle illumination light guide portion 306. In some examples, the handle illumination light guide portion 306 can extend partially toward the window 218, such that the handle illumination 212 can propagate in air from an end of the handle illumination light guide portion 306 to the window 218. In some examples, the handle illumination light guide portion 306 can extend fully to the window 218, such that the handle illumination 212 may not propagate in air from the end of the handle illumination light guide portion 306 to the window 218. In some examples, the window 218 can be formed integrally with the handle illumination light guide portion 306. In some examples, the window 218 can be formed as an end of the handle illumination light guide portion 306.

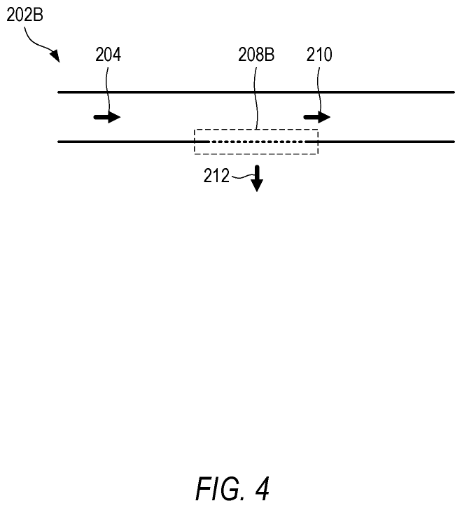

There can optionally be one or more intervening optical elements in the optical path traversed by the handle illumination 212. For example, there can be a spectral filter in the optical path, which can impart a specified color to the handle illumination 212 that emerges from the window 218. Other optical elements can also be used. Such optional optical elements can be used similarly in the configurations of FIGS. 4-6, discussed below.

FIG. 4 shows a side view of another example 208B of an illumination splitter, such as 208 (FIG. 2).

In the configuration of FIG. 4, the light guide 202B is shaped to direct the illumination via total internal reflection from at least one exterior surface of the light guide 202B.

In the configuration of FIG. 4, the illumination splitter 208B can include a textured portion of a surface of the light guide 202B. The textured portion can break the condition of total internal reflection, such that the textured portion can extract the handle illumination 212 from the light guide 202B to propagate in free space toward the window 218.

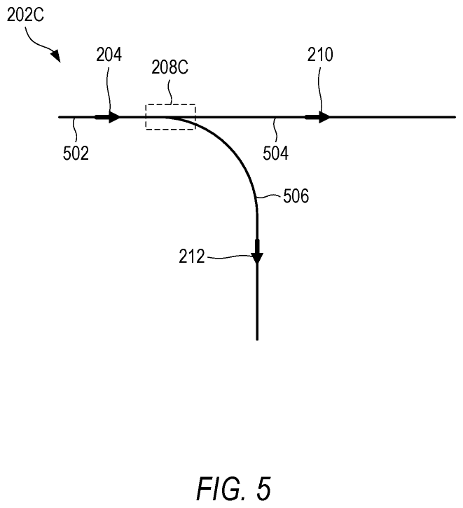

FIG. 5 shows a side view of another example 208C of an illumination splitter, such as 208 (FIG. 2).

In the configuration of FIG. 5, the light guide 202C can include an optical fiber, such as a multi-mode optical fiber or a single-mode optical fiber. Such an optical fiber can guide light in a core of the optical fiber via total internal reflection from a cladding, surrounding the core, of the optical fiber.

In the configuration of FIG. 5, the illumination splitter 208C can include a fiber splitter. The optical fiber can include a received illumination optical fiber portion 502 that can guide the received illumination 204 to the fiber splitter. The optical fiber can include a target illumination optical fiber portion 504, extending from the fiber splitter, that can guide the target illumination 210 along the target illumination optical fiber portion 504. The optical fiber can include a handle illumination optical fiber portion 506, extending from the fiber splitter, that can guide the handle illumination 212 toward the window 218 along the handle illumination optical fiber portion 506. The handle illumination 212 can optionally exit a longitudinal end of the handle illumination optical fiber portion 506, can optionally propagate in free space or in another optical element toward the window 218, can optionally expand in size as it propagates toward the window 218.

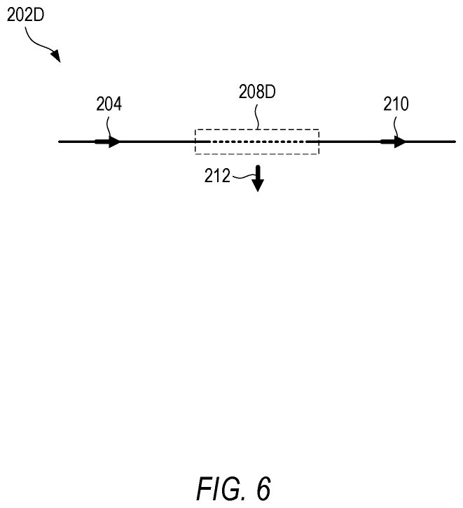

FIG. 6 shows a side view of another example 208D of an illumination splitter, such as 208 (FIG. 2).

In the configuration of FIG. 6, the light guide 202D can include an optical fiber, such as a multi-mode optical fiber or a single-mode optical fiber. Such an optical fiber can guide light in a core of the optical fiber via total internal reflection from a cladding, surrounding the core, of the optical fiber.

In the configuration of FIG. 6, the optical fiber can include a textured surface portion. The textured portion can break the condition of total internal reflection, such that the textured surface portion can extract the handle illumination 212 from the optical fiber to propagate in free space or in another optical element toward the window 218.

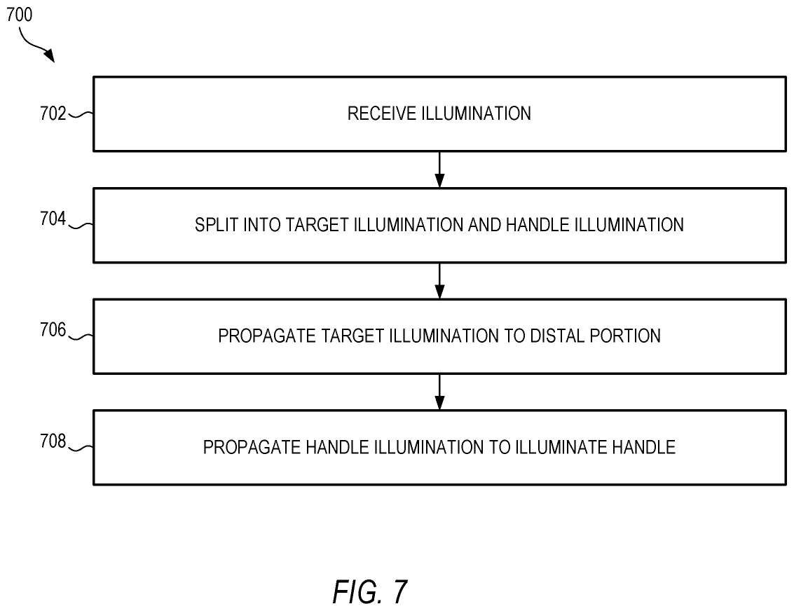

FIG. 7 shows an example of a method for operating an endoscope. The endoscope can include a handle, a shaft extending distally from the handle, and a distal portion at a distal end of the shaft. The method can be executed on the endoscope of FIGS. 1 and 2, or on any other suitable endoscope. The method can be executed with any or all of the illumination configurations shown in FIGS. 3-6. The method is but one example of a method for operating an endoscope. Other suitable methods can also be used.

At operation 702, a practitioner can use the endoscope to receive illumination from a light source that is separate from the endoscope body.

At operation 704, the practitioner can use the endoscope to split the received illumination into a target illumination and a handle illumination.

At operation 706, the practitioner can use the endoscope to propagate the target illumination to the distal portion to illuminate a target.

At operation 708, the practitioner can use the endoscope to propagate the handle illumination to illuminate at least a portion of the handle.

In some examples, the handle can include a window. For these examples, the method can further use the endoscope to pass the handle illumination out of the handle through the window.

In some examples, the illumination can be received with a light guide. The received illumination can be split with a junction in the light guide. The target illumination can be propagated via total internal reflection along a target illumination light guide portion that extends from the junction. The handle illumination light guide portion can propagate toward the window via total internal reflection along a handle illumination light guide portion that extends from the junction.

In some examples, the illumination can be received with a light guide. The received illumination can be split with a textured portion of a surface of the light guide. The handle illumination can be extracted from the light guide by the textured portion and propagates in free space toward the window.

### EXAMPLES

To further illustrate the device, related system, and/or related method discussed herein, a non-limiting list of examples is provided below. Each of the following non-limiting examples can stand on its own or can be combined in any permutation or combination with any one or more of the other examples.

In Example 1, an endoscope can include an endoscope body that includes a handle, a shaft extending distally from the handle, and a distal portion at a distal end of the shaft, the handle including a light guide configured to receive illumination from a light source that is separate from the endoscope body, the light guide including an illumination splitter configured to split the received illumination into a target illumination and a handle illumination, the light guide configured to propagate the target illumination to the distal portion to illuminate a target, the distal portion including a camera configured to capture an image of the illuminated target, the illumination splitter further configured to propagate the handle illumination to illuminate at least a portion of the handle.

In Example 2, the endoscope of Example 1 can optionally be configured such that: the endoscope body includes an instrument sheath that extends through the shaft to the distal portion; the handle includes an instrument port configured to provide access to a proximal end of the instrument sheath; and the illumination splitter is further configured to propagate the handle illumination to illuminate at least a portion of the instrument port.

In Example 3, the endoscope of any one of Examples 1-2 can optionally be configured such that: the distal portion is actuatable; the handle includes a distal portion controller configured to actuate the distal portion; and the illumination splitter is further configured to propagate the handle illumination to illuminate at least a portion of the distal portion controller.

In Example 4, the endoscope of any one of Examples 1-3 can optionally be configured such that: the distal portion is actuatable; the handle includes a distal portion controller configured to actuate the distal portion; and the illumination splitter is further configured to propagate the handle illumination to at least partially illuminate a perimeter of the distal portion controller.

In Example 5, the endoscope of any one of Examples 1-4 can optionally be configured such that: the handle includes indicia that provide visual instructions for operating the endoscope; and the illumination splitter is further configured to propagate the handle illumination to form a backlight for the indicia.

In Example 6, the endoscope of any one of Examples 1-5 can optionally be configured such that: the handle includes indicia that provide visual instructions for operating the endoscope; and the illumination splitter is further configured to propagate the handle illumination to at least partially illuminate a perimeter of the indicia.

In Example 7, the endoscope of any one of Examples 1-6 can optionally be configured such that: the handle includes indicia that identify at least one characteristic of the endoscope; and the illumination splitter is further configured to propagate the handle illumination to form a backlight for the indicia.

In Example 8, the endoscope of any one of Examples 1-7 can optionally be configured such that: the handle includes indicia that identify at least one characteristic of the endoscope; and the illumination splitter is further configured to propagate the handle illumination to at least partially illuminate a perimeter of the indicia.

In Example 9, the endoscope of any one of Examples 1-8 can optionally be configured such that the handle includes a window configured to pass the handle illumination out of the handle.

In Example 10, the endoscope of any one of Examples 1-9 can optionally be configured such that the light guide is shaped to direct the illumination via total internal reflection from at least one exterior surface of the light guide.

In Example 11, the endoscope of any one of Examples 1-10 can optionally be configured such that: the illumination splitter comprises a junction in the light guide; the light guide includes a received illumination light guide portion configured to guide the received illumination to the junction; the light guide includes a target illumination light guide portion extending from the junction and configured to guide the target illumination along the target illumination light guide portion via total internal reflection from at least one exterior surface of the target illumination light guide portion; and the light guide includes a handle illumination light guide portion extending from the junction and configured to guide the handle illumination toward the window along the handle illumination light guide portion via total internal reflection from at least one exterior surface of the handle illumination light guide portion.

In Example 12, the endoscope of any one of Examples 1-11 can optionally be configured such that the illumination splitter comprises a textured portion of a surface of the light guide, the textured portion configured to extract the handle illumination from the light guide to propagate in free space toward the window.

In Example 13, the endoscope of any one of Examples 1-12 can optionally be configured such that the light guide includes an optical fiber.

In Example 14, the endoscope of any one of Examples 1-13 can optionally be configured such that: the illumination splitter comprises a fiber splitter; the optical fiber includes a received illumination optical fiber portion configured to guide the received illumination to the fiber splitter; the optical fiber includes a target illumination optical fiber portion extending from the fiber splitter and configured to guide the target illumination along the target illumination optical fiber portion; and the optical fiber includes a handle illumination optical fiber portion extending from the fiber splitter and configured to guide the handle illumination toward the window along the handle illumination optical fiber portion.

In Example 15, the endoscope of any one of Examples 1-14 can optionally be configured such that the optical fiber includes a textured surface portion, the textured surface portion configured to extract the handle illumination from the optical fiber to propagate in free space toward the window.

In Example 16, a method for operating an endoscope, the endoscope including an endoscope body, the endoscope body including a handle, a shaft extending distally from the handle, and a distal portion at a distal end of the shaft, can include: receiving illumination from a light source that is separate from the endoscope body; splitting the received illumination into a target illumination and a handle illumination; propagating the target illumination to the distal portion to illuminate a target; and propagating the handle illumination to illuminate at least a portion of the handle.

In Example 17, the method of claim 16 can optionally be configured such that the handle includes a window, and further comprising passing the handle illumination out of the handle through the window.

In Example 18, the method of any one of claims 16-17 can optionally be configured such that: the illumination is received with a light guide; the received illumination is split with a junction in the light guide; the target illumination is propagated via total internal reflection along a target illumination light guide portion that extends from the junction; and the handle illumination light guide portion is propagated toward the window via total internal reflection along a handle illumination light guide portion that extends from the junction.

In Example 19, the method of any one of claims 16-18 can optionally be configured such that: the illumination is received with a light guide; the received illumination is split with a textured portion of a surface of the light guide; and the handle illumination is extracted from the light guide by the textured portion and propagates in free space toward the window.

In Example 20, an endoscope can include: an endoscope body that includes a handle that includes a window, a shaft extending distally from the handle, and a distal portion at a distal end of the shaft, the handle including a light guide configured to receive illumination from a light source that is separate from the endoscope body, the light guide including a textured portion configured to split the received illumination into a target illumination and a handle illumination, the light guide configured to propagate the target illumination to the distal portion to illuminate a target, the distal portion including a camera configured to capture an image of the illuminated target, the textured portion configured to propagate the handle illumination toward the window to illuminate at least a portion of the handle.

What is claimed is:

1. An endoscope, comprising:
an endoscope body that includes a handle, a shaft extending distally from the handle, and a distal portion at a distal end of the shaft,
the handle including a light guide configured to receive illumination from a light source that is separate from the endoscope body,
the light guide including an illumination splitter configured to split the received illumination into a target illumination and a handle illumination,
the light guide configured to propagate the target illumination to the distal portion to illuminate a target,
the distal portion including a camera configured to capture an image of the illuminated target,
the illumination splitter further configured to propagate the handle illumination to illuminate at least a portion of the handle.

2. The endoscope of claim 1, wherein:
the endoscope body includes an instrument sheath that extends through the shaft to the distal portion;
the handle includes an instrument port configured to provide access to a proximal end of the instrument sheath; and
the illumination splitter is further configured to propagate the handle illumination to illuminate at least a portion of the instrument port.

3. The endoscope of claim 1, wherein:
the distal portion is actuatable;
the handle includes a distal portion controller configured to actuate the distal portion; and
the illumination splitter is further configured to propagate the handle illumination to illuminate at least a portion of the distal portion controller.

4. The endoscope of claim 1, wherein:
the distal portion is actuatable;
the handle includes a distal portion controller configured to actuate the distal portion; and
the illumination splitter is further configured to propagate the handle illumination to at least partially illuminate a perimeter of the distal portion controller.

5. The endoscope of claim 1, wherein:
the handle includes indicia that provide visual instructions for operating the endoscope; and
the illumination splitter is further configured to propagate the handle illumination to form a backlight for the indicia.

6. The endoscope of claim 1, wherein:
the handle includes indicia that provide visual instructions for operating the endoscope; and
the illumination splitter is further configured to propagate the handle illumination to at least partially illuminate a perimeter of the indicia.

7. The endoscope of claim 1, wherein:
the handle includes indicia that identify at least one characteristic of the endoscope; and
the illumination splitter is further configured to propagate the handle illumination to form a backlight for the indicia.

8. The endoscope of claim 1, wherein:
the handle includes indicia that identify at least one characteristic of the endoscope; and
the illumination splitter is further configured to propagate the handle illumination to at least partially illuminate a perimeter of the indicia.

9. The endoscope of claim 1, wherein the handle includes a window configured to pass the handle illumination out of the handle.

10. The endoscope of claim 9, wherein the light guide is shaped to direct the illumination via total internal reflection from at least one exterior surface of the light guide.

11. The endoscope of claim 10, wherein:
the illumination splitter comprises a junction in the light guide;
the light guide includes a received illumination light guide portion configured to guide the received illumination to the junction;
the light guide includes a target illumination light guide portion extending from the junction and configured to guide the target illumination along the target illumination light guide portion via total internal reflection from at least one exterior surface of the target illumination light guide portion; and
the light guide includes a handle illumination light guide portion extending from the junction and configured to guide the handle illumination toward the window along the handle illumination light guide portion via total internal reflection from at least one exterior surface of the handle illumination light guide portion.

12. The endoscope of claim 10, wherein the illumination splitter comprises a textured portion of a surface of the light guide, the textured portion configured to extract the handle illumination from the light guide to propagate in free space toward the window.

13. The endoscope of claim 9, wherein the light guide includes an optical fiber.

14. The endoscope of claim 13, wherein:
the illumination splitter comprises a fiber splitter;
the optical fiber includes a received illumination optical fiber portion configured to guide the received illumination to the fiber splitter;
the optical fiber includes a target illumination optical fiber portion extending from the fiber splitter and configured to guide the target illumination along the target illumination optical fiber portion; and
the optical fiber includes a handle illumination optical fiber portion extending from the fiber splitter and configured to guide the handle illumination toward the window along the handle illumination optical fiber portion.

15. The endoscope of claim 13, wherein the optical fiber includes a textured surface portion, the textured surface portion configured to extract the handle illumination from the optical fiber to propagate in free space toward the window.

16. A method for operating an endoscope, the endoscope including an endoscope body, the endoscope body including a handle, a shaft extending distally from the handle, and a distal portion at a distal end of the shaft, the method comprising:
receiving illumination from a light source that is separate from the endoscope body;
splitting the received illumination into a target illumination and a handle illumination;
propagating the target illumination to the distal portion to illuminate a target; and
propagating the handle illumination to illuminate at least a portion of the handle.

17. The method of claim 16, wherein the handle includes a window, and further comprising passing the handle illumination out of the handle through the window.

18. The method of claim 17, wherein:
the illumination is received with a light guide;
the received illumination is split with a junction in the light guide;
the target illumination is propagated via total internal reflection along a target illumination light guide portion that extends from the junction; and
the handle illumination light guide portion is propagated toward the window via total internal reflection along a handle illumination light guide portion that extends from the junction.

19. The method of claim 17, wherein:
the illumination is received with a light guide;
the received illumination is split with a textured portion of a surface of the light guide; and
the handle illumination is extracted from the light guide by the textured portion and propagates in free space toward the window.

20. An endoscope, comprising:
an endoscope body that includes a handle that includes a window, a shaft extending distally from the handle, and a distal portion at a distal end of the shaft,
the handle including a light guide configured to receive illumination from a light source that is separate from the endoscope body,
the light guide including a textured portion configured to split the received illumination into a target illumination and a handle illumination,
the light guide configured to propagate the target illumination to the distal portion to illuminate a target,
the distal portion including a camera configured to capture an image of the illuminated target,
the textured portion configured to propagate the handle illumination toward the window to illuminate at least a portion of the handle.
